(19) United States
(12) Patent Application Publication
(10) Pub. No.: US 2026/0249042 A1
(43) Pub. Date: Aug. 27, 2026

(54) SYSTEMS AND METHODS OF FORMING A REINFORCED CATHETER SHAFT

(71) Applicant: Merit Medical Systems, Inc., South Jordan, UT (US)

(72) Inventor: David Christian Lentz, Sandy, UT (US)

(21) Appl. No.: 19/548,206

(22) Filed: Feb. 24, 2026

Related U.S. Application Data
(60) Provisional application No. 63/762,879, filed on Feb. 25, 2025.

(51) Int. Cl. A61M 25/00

(52) U.S. Cl. CPC A61M 25/0012; A61M 25/0045; A61M 2207/00

(57) ABSTRACT

The present disclosure describes systems and methods of forming a continuous reinforced catheter shaft having multiple coiled wire structures. A method includes providing of a first spool having a liner rolled thereon and unrolling at least a portion of the liner from the first spool. The method also includes coiling a reinforcing wire on at least the portion of the liner unrolled from the first spool to form a plurality of coiled wire structures and an interim portion of the reinforcing wire coiled adjacent coiled wire structures. The method also includes securing a jacket over the plurality of coiled wire structures and the interim portion of the reinforcing wire coiled to form a continuous shaft having the plurality of coiled wire structures.

### RELATED APPLICATIONS

This application claims priority to U.S. Provisional Application No. 63/762,879, filed on Feb. 25, 2025 and titled, “Systems and Methods of Forming a Reinforced Catheter Shaft,” which is hereby incorporated by reference in its entirety.

### TECHNICAL FIELD

The present disclosure relates generally to medical sheaths including introducer sheaths and methods to manufacture introducer sheaths. More particularly, some embodiments relate to systems and methods of forming a reinforced catheter shaft including coiled wire structures.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1A illustrates a side view of a portion of a sheath, according to an embodiment of the present disclosure.

FIG. 1B illustrates a cross-sectional view of the portion of the sheath of FIG. 1A, according to an embodiment of the present disclosure.

FIG. 1C illustrates a block diagram of a catheter coil winding system in a first phased of winding, according to an embodiment of the present disclosure.

FIG. 1D illustrates a block diagram of a catheter coil winding system of FIG. 1C, in a second phase of winding, according to an embodiment of the present disclosure.

FIG. 1E illustrates a lateral view of a sheath portion as formed by the catheter coil winding system of FIGS. 1C-D, according to an embodiment of the present disclosure.

FIG. 2 illustrates a perspective view of an introducer sheath with a sheath including a coiled wire structure, according to one embodiment of the present disclosure.

FIG. 3A illustrates a perspective component view of an introducer sheath assembly, according to one embodiment of the present disclosure.

FIG. 3B illustrates a perspective view of the introducer sheath assembly of FIG. 3A, assembled for use, according to one embodiment of the present disclosure.

FIG. 4 illustrates a flow diagram of a method of manufacturing an introducer sheath, according to one embodiment of the present disclosure.

### DETAILED DESCRIPTION

A support catheter is a medical device used to provide structural support and facilitate the insertion of other medical instruments into a patient's vasculature. Support catheters are designed to aid in navigation for the placement of other devices, such as guidewire navigation used to deliver stents or angioplasty balloons, within the vasculature. For example, in an instance where a guidewire lacks the necessary stiffness to navigate a lesion, a support catheter can be introduced around the guidewire, offering additional support to cross the obstacle. Once the lesion is traversed, the support catheter can be withdrawn, allowing the guidewire to resume navigation.

There are some requirements for the characteristics of a sheath of a support catheter sheath. For instance, the material should possess sufficient flexibility to navigate through tortuous vascular pathways while maintaining the necessary rigidity to provide structural support during medical procedures. Additionally, the sheath must be able to withstand the forces exerted during insertion and manipulation without buckling or fracturing. The sheath's tensile strength, torsional strength, rigidity, and resistance to kinking directly impact its ability to effectively support and guide other medical instruments within the body.

To meet these requirements, conventional support catheters typically employ combination of a Teflon liner and a braided wire structure in their design. The Teflon liner often serves as an inner layer, providing lubricity to facilitate smooth device advancement within the body's vasculature while also offering a barrier between the wire structure and bodily tissues to minimize friction and potential damage. The braided wire structure provides mechanical strength and flexibility required for navigating through anatomical pathways.

Such a design within conventional support catheters face some challenges. For instance, despite its intended functionality, the braided wire structure can exhibit a degree of flexibility that can lead to complications. For instance, due to frequent, or sharp, bending of the sheath while traversing vasculature, the flexibility of the braided structure can result in the constriction of the inner diameter of the sheath, impeding smooth passage of medical devices, causing difficulty for the operator, or causing discomfort to the patient. Through constriction and friction, the braided structure can compromise the operator's control over the wire, making precise navigation more challenging and increasing the risk of vessel injury.

Aspects and implementations of the present disclosure address these and other challenges by providing a catheter sheath with coiled wire structures. The provided coiled wire structure can include a doubling back, doubling over, or multiple coils, which enhances tensile strength and torqueability, while reducing and/or eliminating changes in diameter and inner diameter constriction during deformation and bending.

A method for forming such a coiled wire structure is also described. For instance, this disclosure describes systems and methods of forming a reinforced catheter sheath having multiple coils.

In some embodiments, the coiled wire structure can include a first coiled wire structure, or coil, traversing the length of the sheath in a first direction. At a distal portion, the wire structure can be coiled back over the first coiled wire structure, or coil, in a second, or opposite direction. The second coil can again traverse the length of the sheath. Otherwise stated, the coiled wire structure can be such that there are two coils overlapping one another along the length of the sheath.

In another example, the coiled wire structure can include a coil where the wire is coiled more densely (e.g., more picks/inch) at the distal portion than elsewhere on the coiled wire structure. According to many embodiments, the coiled wire structure can include a radiopaque material. In embodiments, the distal portion can have one or more coils of the coiled wire structure more densely disposed therein (e.g., double coiled or greater picks/inch braid) may be function as a radiopaque marker during use of the introducer sheath. For example, the distal portion of the coiled wire structure may be used for fluoroscopy identification. While embodiments herein refer to introducer sheaths, the same features may be included on other types of sheaths such as guiding sheaths.

Systems and methods of this disclosure also include medical sheaths with a coiled wire structure having an annealed segment disposed at least proximate to a distal portion of the coiled wire structure. This disclosure also describes methods to manufacture such an introducer sheath. For example, electrodes secured to two pulleys in a catheter coil winding system may be activated to apply a charge or voltage to a segment of reinforcing wire extending between the two pulleys just before the segment of reinforcing wire is coiled around a liner or elongate member. Applying the charge to the segment of reinforcing wire heats the segment of reinforcing wire effective to anneal the segment of reinforcing wire. The electrodes also may be deactivated such that the segment of reinforcing wire between the pulleys is generally the only portion of reinforcing wire that is annealed (e.g., the annealed segment is between two unannealed portions of the reinforcing wire).

Coiled metal structures may also have a desirable spring temper that resists kinks and/or increases the capacity of the shaft to temporarily elastically deform, then spring back without creating a permanent kink. This, in turn, reduces instances where a kinked or deformed introducer shaft must be removed and replaced during a therapy.

Furthermore, during bending or deformation, a coiled wire structure can resist ovalization of the inner lumen of the sheath, when compared to other wired structures.

The annealed segment of the reinforcing wire may form at least some of the distal portion of a coiled wire structure. This annealed segment reduces a tendency of the coiled wire structure to unravel along the annealed portion, while the coiled wire structure maintains a desirable spring temper along the unannealed portions. Also described herein are systems and methods for annealing only selected segments of a reinforcing wire immediately before the reinforcing wire is coiled around a liner, while portions of the reinforcing wire adjacent to the annealed segment remain unannealed.

Introducer sheaths having wire more densely disposed in the distal portion of a coiled wire structure may be manufactured using the methods described in more detail below. In some embodiments, one or more metal wires are coiled or wound on a spring winder from a proximal end to a distal end, then further coiled or wound from the distal end only partially towards the proximal end to form the distal portion having wire more densely disposed than elsewhere in the coiled wire structure. A liner and a jacket are reflowed or melt-bonded onto the frame, and a hub is overmolded around a proximal portion of the reflowed shaft. These methods may be done along a length sufficient to produce a plurality of introducer sheaths. The nylon core may be heated and stretched to reduce the diameter of the nylon core for removal of the frame. A hub may be overmolded or otherwise secured around a proximal portion of the sheath shafts.

The phrase “coupled to” is broad enough to refer to any suitable coupling or other form of interaction between two or more entities, including mechanical, fluidic and thermal interaction. Thus, two components may be coupled to each other even though they are not in direct contact with each other. The phrase “fluid communication” is used in its ordinary sense, and is broad enough to refer to arrangements in which a fluid (e.g., a gas or a liquid) can flow from one element to another element when the elements are in fluid communication with each other.

The terms “proximal” and “distal” are opposite directional terms. As used herein, the distal end of a device or component is the end of the component that is furthest from the physician during ordinary use. The proximal end refers to the opposite end, or the end nearest the physician during ordinary use. For example, the proximal end of an introducer sheath used in minimally invasive vascular treatment is the end accessible to a practitioner during use, while the distal end is disposed within a patient's vascular system when the sheath is placed into such a patient.

An assembler may be any person, system, or machine used in the manufacture of the introducer sheaths.

Embodiments may be understood by reference to the drawings, wherein like parts are designated by like numerals throughout. The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

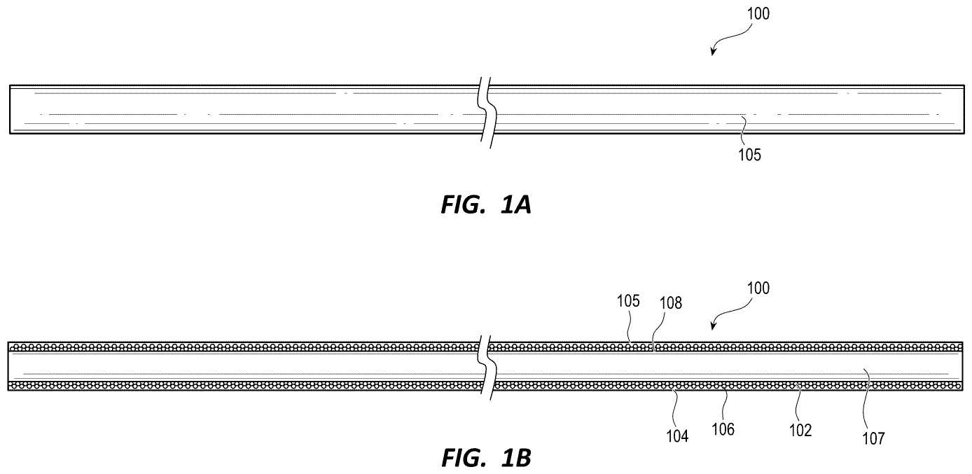

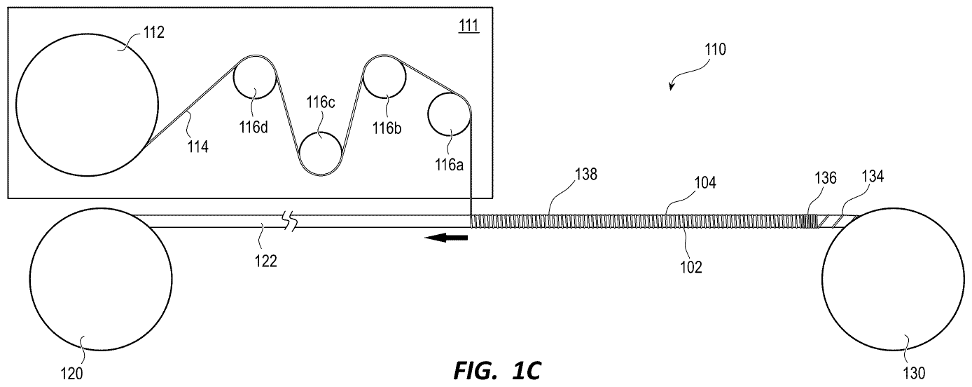

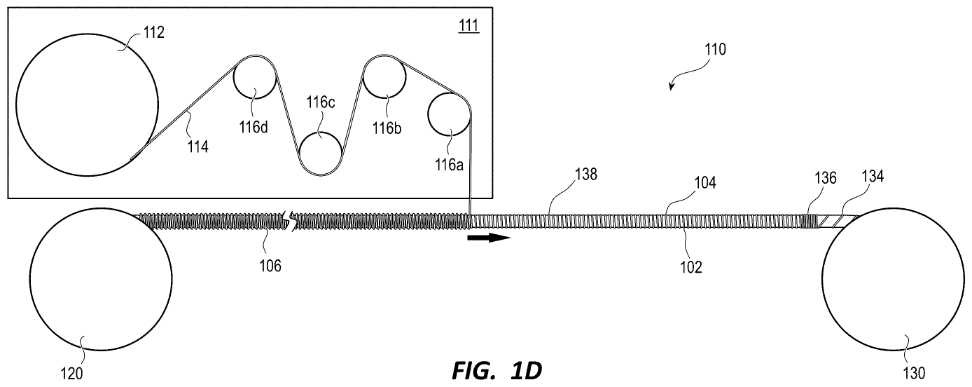

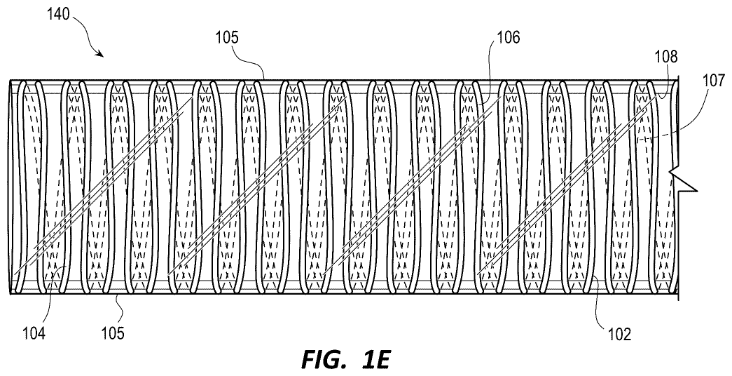

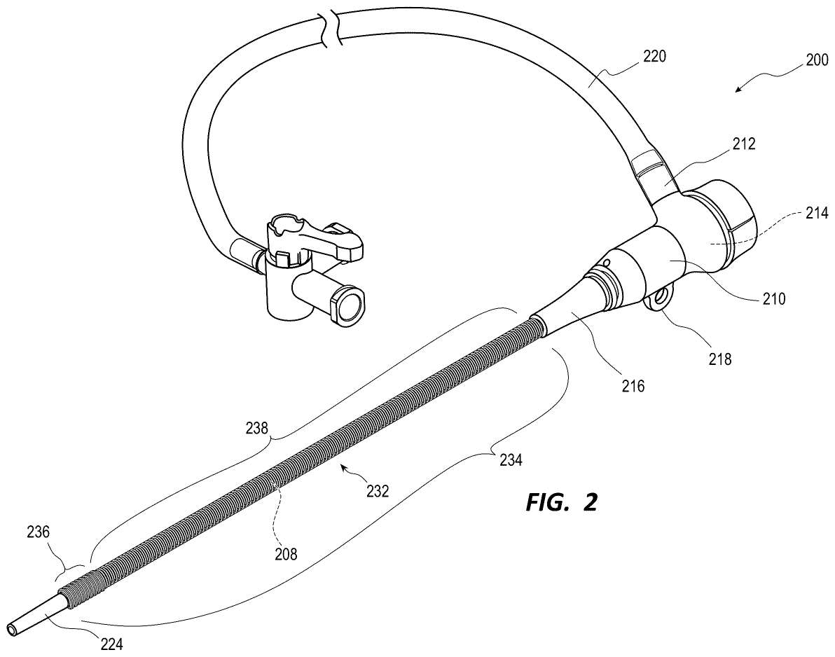

FIGS. 1A-B illustrate a lateral view of a portion of a catheter sheath 100, according to an embodiment of the present disclosure. FIG. 1A illustrates a lateral view of a portion of a sheath 100, according to an embodiment of the present disclosure. FIG. 1B illustrates a cross-sectional view of the portion of the sheath 100 of FIG. 1A, according to an embodiment of the present disclosure. In the illustrated embodiments, the portion of the sheath 100 can including an exterior jacket 105, a coiled wire structure 104, an inner liner 108, and a fluid-conductive lumen 107 disposed therein

The material of the liner 108 can include polyamide resins configured to seal the interior surface of the coiled wire structure 104. The liner 108 can include a surface comprising a lubricious polymeric material. For example, the material can comprise any bio-compatible material having low frictional properties (e.g., TEFLON®, PTFE, fluorinated ethylene propylene (FEP), polyethylene, polyamide, ethylene chlorotrifluoro-ethylene, ethylene tetrafluoroethylene, PVDF).

In some cases, coiled wire structure 104 can include a first coil (e.g., coil 102) and a second coil (e.g., coil 106). Coil 102 can be coiled in a first direction and disposed beneath coil 106. Coil 106 can be coiled in a second direction.

FIGS. 1C-D illustrate a simplified block diagram of a catheter coil winding system 110, that schematically shows a process for coiling the reinforcing wire 114 around the elongate member 122, according to embodiments disclosed herein. FIG. 1C illustrates a block diagram of a catheter coil winding system in a first phased of winding, according to an embodiment of the present disclosure. FIG. 1D illustrates a block diagram of a catheter coil winding system of FIG. 1C, in a second phase of winding, according to an embodiment of the present disclosure. In the illustrated embodiments, the catheter coil winding system 110 can be configured to form one or more coiled wire structure(s) 104 on a single elongate member 122.

To form the coiled wire structure 104, the catheter coil winding system 110 can include a spool holder configured to hold a first spool 120 of the elongate member 122. Elongate member 122 can include a mandrel or a stretchable wire that acts as a mandrel. In embodiments, a liner (e.g., liner 108 of FIG. 1B) is disposed around an exterior circumference of the mandrel. For example, elongate member 122 can be formed by mandrel that can be a metal wire that can include a copper wire, such as a silver-plated copper wire. The first spool 120 can include continuous elongate member 122 rolled onto or otherwise wrapped around the spool 120. Spool 130 can include elongate member 122 rolled onto or otherwise wrapped around the spool 130. A coiled wire structure can be deposited onto a portion of elongate member 122 suspended between spool 120 and 130.

In some cases, after a manufacturing process of the sheath, the mandrel can be removed, leaving behind the liner of the elongate member 122. In such cases, the exterior liner of the elongate member 122 can become the interior liner of the sheath that is formed (e.g., such as liner 108 of FIG. 1B). This interior liner can form an exterior wall to a lumen of the sheath (e.g., lumen 107 of FIG. 1B) formed by the absence of the mandrel.

To facilitate separation of the mandrel of elongate member 122, in some embodiments, the mandrel or metal wire can be coated with low friction materials to ease loading and removal of the liner and mandrel. For example, in some embodiments, the low surface friction mandrel may be a polytetrafluoroethylene (PTFE)-coated metal wire to slide a stainless steel metal frame onto. In embodiments, lubricants may be used to reduce the friction on the surface of the low surface friction metal wire or mandrel of elongate member 122.

The catheter coil winding system 110 can include a spool holder configured to hold a spool 112 of reinforcing wire 114. The reinforcing wire 114 may include tempered steel or brass wires configured to provide a desired spring temper induced to increase their upper limit of elasticity in the coiled wire structure 104. In some embodiments, steels used in the reinforcing wire 114 may include stainless steel, low-alloy, medium-carbon steel, nitinol, and high-carbon steel including those with high yield strength. The spring temper of the reinforcing wire 114 can allow the coiled wire structure 104 to return to its original shape despite deflection, deformation, and/or twisting. In some embodiments, the reinforcing wire 114 may include flat wires. In other embodiments, the reinforcing wire may include round wires.

In some embodiments, catheter coil winding system 110 can include one or more pulleys 116A-D positioned along the reinforcing wire 114 between the spool holder (e.g., the spool 112 of reinforcing wire 114) and the elongate member 122. The one or more pulleys 116A-D can be positioned and configured to have the reinforcing wire 114 wind around a portion of each of the one or more pulleys 116A-D before being coiled around the elongate member 122.

In the catheter coil winding system 110 shown in FIGS. 1C-D, the catheter coil winding system 110 includes four pulleys 116A-D positioned and configured to have the reinforcing wire 114 wind around a portion of each of the four pulleys 116A-D. Other embodiments, however, may include fewer (e.g., one, two, or three) pulleys or more (e.g., five, six, seven, and so on) pulleys than the four pulleys 116A-D shown in FIG. 1C-D.

In embodiments, the catheter coil winding system 110 includes an assembly 111 including the spool 112 of reinforcing wire 114 and the one or more pulleys 116A-D. The assembly 111 is configured to rotate around the elongate member 122 to coil the reinforcing wire 114 around the elongate member 122. In other words, the catheter coil winding system 110 is configured such that one or more (e.g., all) of the spool 112 of reinforcing wire 114 and the one or more pulleys 116A-D rotate around the elongate member 122 to coil the reinforcing wire 114 around at least a portion of the elongate member 122.

Catheter coil winding system 110 can coil wire about a portion of elongate member 122 suspended between first spool 120 and second spool 130. As seen in FIG. 1C, the suspended portion can be translated in a first direction, as the first coil 102 is formed. First spool 120 and second spool 130 (or corresponding chucks) can be rotated, or translated, in a first direction, to form the first coil 102. As seen in FIG. 1D, once the first coil 102 has been disposed on to the liner, a second coil 106 can be disposed onto the first coil 102. In embodiments, the suspended portion can be translated in a second direction, opposite the first direction, as the second coil 106 is formed. First spool 120 and second spool 130 (or corresponding chucks) can be rotated, or translated, in a second direction, to form the second coil 106.

In some embodiments, the first spool 120 is positioned or positionable on the spool holder of the catheter coil winding system 110 such that the elongate member 122 may be unrolled from the first spool 120 to extend past one or more pulleys 116A-D for coiling of the reinforcing wire 114 thereon.

In many embodiments, as one or more (e.g., all) of the spool 112 of reinforcing wire 114 and the one or more pulleys 116A-D rotate around the elongate member 122 to coil the reinforcing wire on the elongate member 122, the elongate member 122 may be pulled from first spool 120 and/or second spool 130 at variable rates. For instance, in embodiments, during formation of the first coil 102, elongate member 122 can be pulled from the first spool 120 at a first rate when a proximal portion 138 is being formed and at a second rate slower than the first rate when the distal portion 136 is being formed. In some cases, pulling the elongate member 122 from the first spool 120 at the first rate and the second rate can be effective for forming the distal portion 136 having the reinforcing wire 114 coiled more densely thereon than the proximal portion 138 of the coiled wire structure 104. Variable rates can be similarly used during formation of the second coil 106.

In some embodiments, as one or more (e.g., all) of the spool 112 of reinforcing wire 114 and the one or more pulleys 116A-D rotate around the elongate member 122 to coil the reinforcing wire on the elongate member 122, the elongate member 122 can be pulled from the first spool 120 and/or second spool 130 at any number of varied rates. For instance, in embodiments, elongate member 122 can be pulled at a first rate when a proximal portion 138 is being formed, at a second rate slower than the first rate when the distal portion 136 is being formed. In some embodiments, the elongate member 122 can be pulled at a third rate, to form more loosely coiled, interim, and/or extraneous portions, as seen by portion 134.

In some embodiments, the elongate member 122 can be secured to second spool 130, distal to the first spool 120. The first spool 120 and the second spool 130 can be positioned such that reinforcing wire 114 pulled from the spool 112 can be coiled around the elongate member 122 extending between the first spool 120 and the second spool 130. For example, pulley 116A can be positioned between first spool 120 and second spool 130. Second spool 130 can be positioned on a second spool holder configured to rotate the second spool 130 around an axis of the second spool 130 effective to pull the elongate member 122 from the first spool 120. More specifically, the second spool holder can be configured to rotate the second spool 130 around the axis of the second spool 130 at different rates, and effectively pull the elongate member 122 from the first spool 120 at the first rate, the second rate, and/or a third rate to form a proximal portion, a distal portion, and/or as many additional variable-rate portions as is feasible or appropriate) onto elongate member 122. Elongate member 122 can be secured to second spool 130 such that as the second spool holder rotates the second spool 130, the elongate member 122 having the coiled wire structure 104 is rolled around the second spool 130.

In some embodiments, the catheter coil winding system 110 can include a proximal chuck rather than the first spool 120 and/or a distal chuck rather than the second spool 130. In other words, in some embodiments, the circle 120 can represent a proximal chuck and the circle 130 can represent a distal chuck. The proximal chuck and the distal chuck can be configured to secure to the elongate member 122 and can be spaced from one another at a distance for a plurality of coiled wire structures to be formed on the elongate member 122 between the proximal chuck and the distal chuck. In some embodiments, the proximal chuck and/or the distal chuck can be configured to move simultaneously as the reinforcing wire 114 is being coiled around the suspended portion of the elongate member 122 effective to form the proximal portion 138, the distal portion 136. For example, with the elongate member 122 being secured thereto, the proximal chuck and/or the distal chuck can be configured to move the elongate member 122: at a first rate as the reinforcing wire 114 is being coiled around the elongate member 122 to form a proximal portion 138; and at a second rate slower than the first rate as the reinforcing wire 114 is being coiled around the elongate member 122 to form a distal portion 136. In some embodiments of the catheter coil winding system 110, the proximal chuck may replace the first spool holder/first spool 120, while the second spool holder/second spool 130 remains. In some embodiments, the distal chuck may replace the second spool holder/second spool 130, while the first spool holder/first spool 120 remain.

In some embodiments, the catheter coil winding system 110 can be configured to anneal selected segments of reinforcing wire 114 pulled from the spool 112 just prior to (e.g. immediately before) the reinforcing wire 114 is wound around the elongate member 122 including the liner 108. In other words, the catheter coil winding system 110 may include an “in-line” annealing system. For example, the catheter coil winding system 110 can also include an annealing assembly configured to selectively anneal segments of the reinforcing wire between the spool holder and the elongate member 122.

In some embodiments, the annealing assembly includes the one or more pulleys 116A-D positioned along the reinforcing wire 114 between the spool holder (e.g., the spool 112 of reinforcing wire 114) and the elongate member 122. In some embodiments, the one or more pulleys 116A-D can be at least partially metal. For example, the one or more pulleys 116A-D may include a rotatable metal disc or cylinder that the reinforcing wire 114 at least partially contacts. The catheter coil winding system 110 can also include an electrode secured to at least one pulley of the one or more pulleys 116A-D and an actuator configured to activate and deactivate the electrode. In some embodiments, the catheter coil winding system 110 includes two electrodes each secured to a different pulley of the pulleys 116A-D. For example, the catheter coil winding system 110 can include a first electrode secured to a first pulley 116A and a second electrode secured to a second pulley 116B. In some embodiments, the catheter coil winding system 110 can include multiple electrodes each coupled a different pulley of the multiple pulleys 116A-D.

As noted above, the actuator is configured to selectively activate and deactivate the electrodes. In some embodiments, the electrode may be secured to a pulley of the multiple pulleys 116A-D such that the electrode applies a voltage or charge to the metal disc or cylinder of the pulley when the electrode is activated. Activating the electrodes is effective to anneal a segment of the reinforcing wire 114 before the segment of the reinforcing wire 114 is coiled around at least the portion of the elongate member 122 (e.g., after the reinforcing wire 114 has been unspooled from the spool 112, but before the reinforcing wire 114 has been wound around the elongate member 122). For example, when the catheter coil winding system 110 include the first electrode secured to the first pulley 116A and the second electrode secured to the second pulley 116B, activating the electrodes secured to the first pulley 116A and the second pulley 116B anneals the segment of the reinforcing wire 114 that extends between the first pulley 116A and the second pulley 116B. More specifically, activating the electrodes secured to the first pulley 116A and the second pulley 116B can apply a voltage or charge to the segment of the reinforcing wire 114 positioned between the first pulley 116A and the second pulley 116B effective to heat and anneal the segment of the reinforcing wire 114 that extends between the first pulley 116A and the second pulley 116B.

In some embodiments, the two pulleys (e.g., pulleys 116A, 116B) having an electrode secured thereto are positioned at a selected and/or adjustable linear distance such that the activation of the electrodes secured to the two pulleys anneals a segment of the reinforcing wire 114 have a selected distance corresponding or correlated to the linear distance between the two pulleys. In embodiments having more than two pulleys, the two pulleys having an electrode secured thereto may be the last two pulleys the reinforcing wire 114 contacts before the reinforcing wire 114 is would around the elongate member 122.

As discussed, within FIG. 1D, after a first coil 102 of reinforcing wire 114 is coiled around the elongate member 122, the direction of travel can be reversed, effective to form an additional portion of the coiled wire structure (e.g., second coil 106). For example, once the direction of travel, as seen in FIG. 1C has reached a proximal end of the coiled wire structure 104, or reached previously-established length or distance of the coiled wire structure 104, the direction of travel can be reversed, and a second coil 106 can be positioned on the elongate member 122. Otherwise stated, second coil 106 can be formed on top of, or radially exterior to, first coil 102. Second coil 106 can be coiled in a second direction, or opposite of the direction of the first coil 102, due to the change in direction of travel.

Thus, in embodiments, coiled wire structure 104 can include multiple wire coils layered onto elongate member 100. In embodiments, each coil (e.g., first coil 102 and/or second coil 106) can include separate portions (e.g., a proximal portion and a distal portion) having the reinforcing wire 114 coiled more densely and/or more tightly than others.

In embodiments, portions along the elongate member 122 can include similarly densities of both the first coil 102 and the second coil 106 along specific portions. For instance, with respect to a the distal portion 136, both first coil 102 and second coil 106 can be more densely coiled at that portion of elongate member 122. In some embodiments, the catheter coil winding system 110 can be configured to dispose the second coil 106, while moving the suspended portion at a rate that is slower than while disposing the first coil 102. Otherwise stated, in embodiments, second coil 106 can be wound or coiled around elongate member 122 with a lower density than first coil 102.

In embodiments including the annealing assembly, the catheter coil winding system 110 can be configured such that the at least some (e.g. most or all) of the distal portion of a coil includes the annealed reinforcing wire 114. For example, in some embodiments, the annealed segment can be located at the distal end of the distal portion 136 when coiled on the elongate member 122. Annealing at least some of the distal portion 136 can remove residual stresses and the spring temper of the coiled wire structure along the annealed portion of the distal portion. For example, annealing can reduce a tendency of the coils to straighten, tending to prevent unraveling. In some embodiments, the catheter coil winding system 110 can be configured to automatically activate and deactivate the electrodes on the first pulley 116A and the second pulley 116B during coiling of the reinforcing wire 114 and formation of the coiled wire structure 104 such that the annealed segment forms at least some of the distal portion 136 of the coiled wire structure 104.

In some embodiments the annealed segment at the distal end of the coiled wire structure 104 may be approximately 1/32 inches long to approximately ¼ inches long, including about 1/16 inches long to about 3/16 inches long and about ⅛ inches long. The length of the annealed portion may vary based on application. In some embodiments, the annealed portion may be approximately 1/32 inches long to approximately ¼ inches long, including about 1/16 inches long to about 3/16 inches long and about ⅛ inches long. In some embodiments, a plurality of portions on the length of a long coiled wire structure 104 may be annealed. The long coiled wire structure 104 can subsequently be cut to form multiple coiled wire structures for inclusion in introducer sheath shafts. The positions of the plurality of annealed portions along the coiled metal frame may thus ultimately correspond to portions that will be disposed as distal ends of a plurality of introducer sheath shafts.

With a distal portion 136 being coiled more tightly than the proximal portion 138, the distal portion 136 can include wire more densely disposed (e.g., tighter pitch) than the proximal portion (e.g., wider pitch). The coiled wire structure 104 can include radiopaque material. For example reinforcing wire 114 can include one or more of tungsten, palladium, platinum, gold, tantalum, depleted uranium, or high radiopaque wire that may be coiled into the coiled wire structure 104 for fluoroscopy identification. In many embodiments, the coiled wire structure 104 consists or consists essentially of the radiopaque material. Accordingly, distal portion 136 may include more densely disposed radiopaque strands of material than the proximal portion 138 of the coiled wire structure 104. The more densely disposed radiopaque strands of material in the distal portion 136 act as a radiopaque marker in fluoroscopy identification, thereby allowing a user to more easily identify the distal region of a sheath shaft during use.

In some embodiments, the distal portion 136 can include a longitudinal length of less than about 125 mm, less than about 100 mm, less than about 75 mm, less than about 50 mm, less than about 25 mm, less than about 10 mm, less than about 5 mm, less than about 3 mm, less, than about 2 mm, less than about 1 mm, about 1 mm to about 125 mm, about 1 mm to about 5 mm, about 5 mm to about 10 mm, about 10 mm to about 25 mm, about 25 mm to about 50 mm, about 50 mm to about 100 mm, about 100 to about 150 mm, about 1 mm, about 2 mm, about 3 mm, about 4, about 5, about 10 mm, about 25 mm, about 50 mm, about 75 mm, or about 100 mm.

In some embodiments, a coiled wire structure can include multiple portions (not shown) having a more dense coil and/or multiple portions having a less dense coil. For example, the coiled wire structure 104 can include the distal portion 136, a first intermediate portion, a second intermediate portion, and/or the proximal portion 138. The first intermediate portion can be disposed between (e.g. adjacent to) the distal portion and the second intermediate portion, and can include a less dense coil than the coil of the distal portion 136 and the second intermediate portion. The second intermediate portion may be disposed between (e.g., adjacent to) the first intermediate portion and the proximal portion 138, and may include a more dense coil than the first intermediate portion and the proximal portion 138.

In these and other embodiments, the distal portion 136 may extend a longitudinal length of less than about 2 mm, the first intermediate portion adjacent the distal portion 136 may extend a longitudinal length of about 3 mm to about 7 mm, the second intermediate portion between first intermediate portion and the proximal portion 138 may extend a longitudinal length about less than about 2 mm.

In use, the distal portion 136 and the second intermediate portion having the denser coil would be visible under fluoroscopy as distinct from the first intermediate portion and the proximal portion 138 having the less dense weave(s). The distance between the distal portion 136 and the second intermediate portion (e.g., the longitudinal length of the first intermediate portion) may be known by the operator and used to measure anatomical features such as a lesion or vessel aneurysm length. Any number or combination of portions with more dense coils may be used. For example, a single device may have two, three, four, or more dense portions (of either or both coils) that may or may not be distributed at even distances from each other. These portions can be configured for measurement of anatomical features, identification of portions of the introducer sheath 100, or both.

FIG. 1E illustrates a lateral view of a sheath portion 140 as formed by the catheter coil winding system of FIGS. 1C-D, according to an embodiment of the present disclosure. In embodiments, sheath portion 140 of the coiled wire structure 104 (as seen and described with respect to FIG. 1E) can be manufactured via the catheter coil winding system 110 (as discussed with respect to FIGS. 1C-D). Accordingly, reference will be made throughout the description of FIG. 1E to specific parts and processes of the disclosure as described with respect to FIGS. 1C-D, whose description and embodiments are incorporated and augmented herein.

In the illustrated embodiment of FIG. 1E, the coiled wire structure 104 can include first coil 102 traversing in a first direction, and second coil 106, traversing the elongate member in a second direction.

In addition to the coiled wire structure 104, the catheter coil winding system 110 may be configured to dispose a jacket 105 over the coiled wire structure 104 and liner 108. In some embodiments, the mandrel portion of the elongate member 122 may be separated from the liner 108 and coiled wire structure 104 prior to the jacket 105 being disposed over the liner 108. In some embodiments, the jacket 105 may be disposed over the liner 108 while the mandrel portion of the elongate member 122 extend through the liner 108. The jacket 105 can have a hydrophilic coating and a larger diameter than the coiled wire structure 104.

The catheter coil winding system 110 can also be configured to dispose an FEP shell (not shown) over the jacket 105. In some embodiments, the mandrel of the elongate member 122 can be removed from the liner 108 prior to the FEP shell being disposed over the jacket 105. In some embodiments, the FEP shell may be disposed over the jacket 105 while the mandrel of the elongate member 122 extends through the liner 108. The FEP shell may have a larger diameter than the jacket 105 and encompass the jacket 105, the coiled wire structure 104, and the liner 108.

The catheter coil winding system 110 can also be configured to apply heat to melt or reflow the liner 108 and jacket 105. Heating the FEP shell can cause it to shrink, reducing its diameter. The reduced diameter of the FEP shell causes the sidewalls of the FEP shell to apply pressure to the encompassed elements (i.e., the jacket 105, the coiled wire structure 104, and the liner 108). The reflow temperature can be within a range that causes the jacket 105 and liner 108 to melt, but not the FEP shell. Thus, the jacket 105 and liner 108 can be reflowed to the coiled wire structure 104 to create a composite conduit forming a sheath portion 140.

According to various embodiments of the catheter coil winding system 110, the jacket 105 may be applied to the liner 108 and coiled wire structure 104 before or after the liner 108 and coiled wire structure 104 are rolled around the second spool 130. According to various embodiments of the catheter coil winding system 110, the FEP shell may be applied to the jacket 105 before or after the liner 108 and coiled wire structure 104 are rolled around the second spool 130.

When the mandrel of the elongate member 122 is removed from the liner 108 (whether before or after the jacket 105 is applied to the liner 108), a sheath lumen 107 is formed in the sheath portion 140.

The sheath portion 140, then, may include the liner 108 defining the sheath lumen 107, the jacket 105, and the coiled wire structure 104 disposed between the jacket 105 and the liner 108. While liner 108, coiled wire structure 104, and jacket 105 are shown as distinct layers in FIGS. 1A-E, the polymer materials of the liner 108 and the jacket 105 may be melted and reflowed together, bonding to the coiled wire structure 104, and each other, and filling any openings in the coiled wire structure 104.

Accordingly, the catheter coil winding system 110 may be configured to form a sheath (e.g., sheath 100 as seen and described in FIG. 1A) comprising a liner 108, a coiled wire structure 104, and a jacket 105 secured over coiled wire structure 104. The coiled wire structure 104 of the sheath may be coiled around the liner 108. The coiled wire structure 104 within the sheath may include a proximal portion and a distal portion having the reinforcing wire 114 of the coiled wire structure coiled with varying densities. The distal portion of the sheath can include a more densely wound reinforcing wire 114.

In some embodiments, the catheter coil winding system 110 may be configured such that the elongate member 122 is rolled on the second spool 130 (e.g., after application of the jacket 105 and/or after the mandrel of elongate member 122 is removed from the liner 108). The sheath portion 140 can then be selectively unrolled from second spool 130. In these and other embodiments, a user may cut multiple sheaths or sheath portions formed on the elongate member 122 to produce one or more sheaths of an appropriate length.

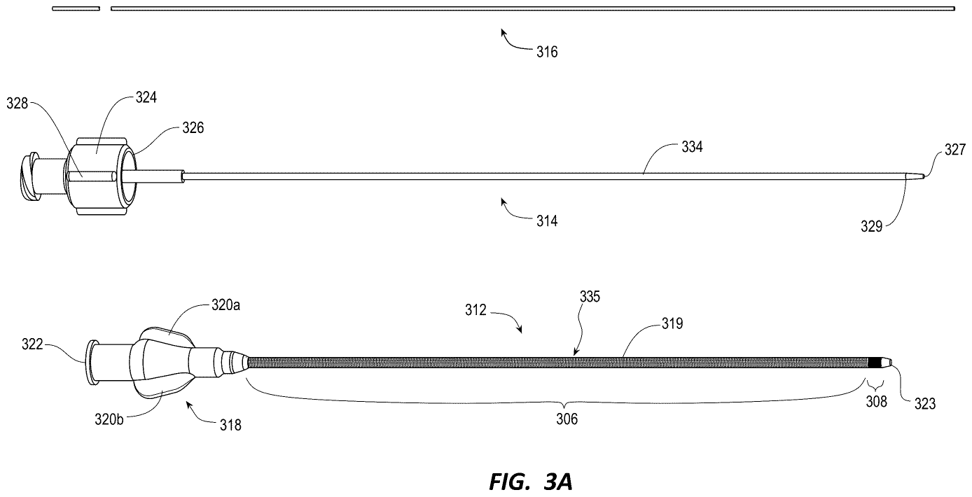

FIG. 2 illustrates a perspective view of an introducer sheath with a sheath including a coiled wire structure, according to one embodiment of the present disclosure. In the illustrated embodiment, an introducer sheath 200 having a sheath 232 formed according to any embodiments of the catheter coil winding system 110 described above. For example, the sheath 232 may include a coiled wire (e.g. metal) structure 234 having a distal portion 236 and a proximal portion 238. Coiled wire structure 234 can include a first coil and a second coil as described above (not labeled in FIG. 2). The first coil can rest underneath the second coil, and be coiled in a different direction than the second coil. Wire from either coil, or any of the wire of the coiled wire structure 234, can be disposed more densely in the distal portion 236 than the proximal portion 238. The coiled wire structure 234, may include an annealed segment, and may be disposed between an interior liner and a jacket (not labeled in FIG. 2). The sheath 232 may be formed according to any of the systems and/or methods described herein.

The sheath 232 may be coupled to and in fluid communication with the hub 210. During some procedures, the hub 210 can be intended to remain exterior of a patient, and the sheath 232 can be intended to at least partially be placed within the vascular system of the patient. Hub 210 can form a chamber that may be accessed via a side port 212 or an introducer bore 214. A suture ring 218 can be coupled to the hub 210 and provide a mechanism allowing a practitioner to grasp the introducer sheath 200 while allowing the introducer sheath 200 to be sutured or fastened to the patient once the introducer sheath 200 has been properly placed. Side port 212 and/or introducer bore 214 can provide entry for medical devices and fluids. For example, a physician may insert a dilator into the introducer bore 214 to assist with placing the introducer sheath 200. The dilator can enter introducer bore 214 through a seal or a valve that maintains hemostasis when the introducer sheath 200 in in communication with the vasculature. Similarly, a fluid channel 220 may couple to the side port 212, establishing a fluid passageway with the chamber of the hub 210. In some embodiments, a sleeve may be used to swage the fluid channel 220 onto the side port 212. A sleeve may be placed over the fluid channel 220 onto the side port 212. Any medical instrument or fluids that enter the chamber of the hub 210 may continue through an opening at the hub distal end 216 into a sheath lumen. Thus, the side introducer bore 214 and a lumen of the fluid channel 220 may both be in fluid communication with a lumen of the sheath 232.

A tip 224 may be coupled to the sheath 232. A cuff (not shown) may overlap the tip-shaft joint to strengthen the joint. The sheath 232 may include the coiled wire structure 234 with an exterior and interior surface coated with a polymer material. In the illustrated embodiment, a liner (not labeled in FIG. 2) can be coupled to the interior surface of the coiled wire structure 234 and define the inside surface of the sheath 232. A jacket (not labeled in FIG. 2) can be coupled to the exterior surface of the coiled wire structure 234 and define the outside surface of the sheath 232.

The introducer sheath 200 may have a thinner wall when compared with traditional introducer sheaths. In some embodiments, the introducer sheath 200 may have an

$$\frac{inner diameter lower limit}{outer diameter upper limit}$$

ratio of greater than 0.85. For example, an introducer sheath for a 4 French (4F) needle or catheter may have an

$$\frac{inner diameter lower limit}{outer diameter upper limit}$$

ratio of greater than 0.85, an introducer sheath for a 5F needle or catheter may have an

$$\frac{inner diameter lower limit}{outer diameter upper limit}$$

ratio of greater than 0.87, an introducer sheath for a 6F needle or catheter may have an

$$\frac{inner diameter lower limit}{outer diameter upper limit}$$

ratio of greater than 0.89, an introducer sheath for a 7F needle or catheter may have an

$$\frac{inner diameter lower limit}{outer diameter upper limit}$$

ratio of greater than 0.90. In some embodiments, the introducer sheath 200 may have an

$$\frac{inner diameter}{outer diameter}$$

ratio ranging between 0.87 and 0.93. For example, an introducer sheath for a 4F needle or catheter may have an average

$$\frac{inner diameter}{outer diameter}$$

ratio ranging between 0.87 and 0.89, an introducer sheath for a 5F needle or catheter may have an average

$$\frac{inner diameter}{outer diameter}$$

ratio ranging between 0.89 and 0.90, an introducer sheath for a 6F needle or catheter may have an average

$$\frac{inner diameter}{outer diameter}$$

ratio ranging between 0.90 and 0.91, and an introducer sheath for a 7F needle or catheter may have an average

$$\frac{inner diameter}{outer diameter}$$

ratio ranging between 0.92 and 0.93.

At least a portion of the coiled wire structure 234 can be annealed and at least a portion of the coiled wire structure 234 may be unannealed. In some embodiments, at least some of the distal portion 236 is annealed and the proximal portion 238 is unannealed. Various aspects and positioning of annealing a coiled wire structure of a sheath are disclosed in U.S. patent Publication Ser. No. 17/457,878, the disclosure of which is incorporated herein, in its entirety, by this reference. The term unannealed refers to portions of the coiled wire structure 234 that retain or otherwise are configured with more spring temper than an annealed portion. Tip 224 may be coupled to the sheath 232 and adjacent an annealed portion of the distal portion 236. In some embodiments, a cuff (not labeled in FIG. 2) may be positioned across the joint between the tip 224 and the annealed portion of the distal portion 236. The cuff may smooth the transition between the tip 224 and the annealed portion of the distal portion 236 and increase the strength of the joint.

The sheath 232 may include the tip 224 coupled to the composite conduit sheath 232 adjacent the distal tip of the coiled wire structure 234. Tip 224 may include or be a polymeric material configured to be malleable to reduce trauma when the introducer sheath enters the vascular system and increase trackability of the introducer over a guidewire. A butt joint may couple the tip 224 to the sheath 232. A butt joint can ensure smoothness through the transition from the tip 224 to the sheath 232. In some embodiments, tip 224 may overlap the sheath 232. In some embodiments, a cuff may overlap tip 224 and the annealed section of the sheath shaft 232 to strengthen the butt joint. Coupling tip 224 to the sheath 232 may be accomplished, for example, via loading the components on a low surface friction mandrel (not necessarily the same low surface friction mandrel discussed above, though it may be). An FEP shell (not necessarily the same FEP shell discussed above) may encompass tip 224 and the sheath 232. Heat may cause the FEP shell to shrink and apply pressure while tip 224 melts to the sheath 232.

The sheath 232 can include an overmolded tip 224. Tip 224 can couple to the sheath shaft 232 at a distal end and can be coupled via an overmolding process. Similarly, hub 210 can be overmolded at a proximal end of the sheath 232. In some embodiments, tip 224 can be more radiopaque than other components to facilitate imaging.

Other embodiments of introducer sheaths including a shaft having a coiled wire structure formed according to embodiments of the catheter coil winding system 110 described above are also disclosed herein.

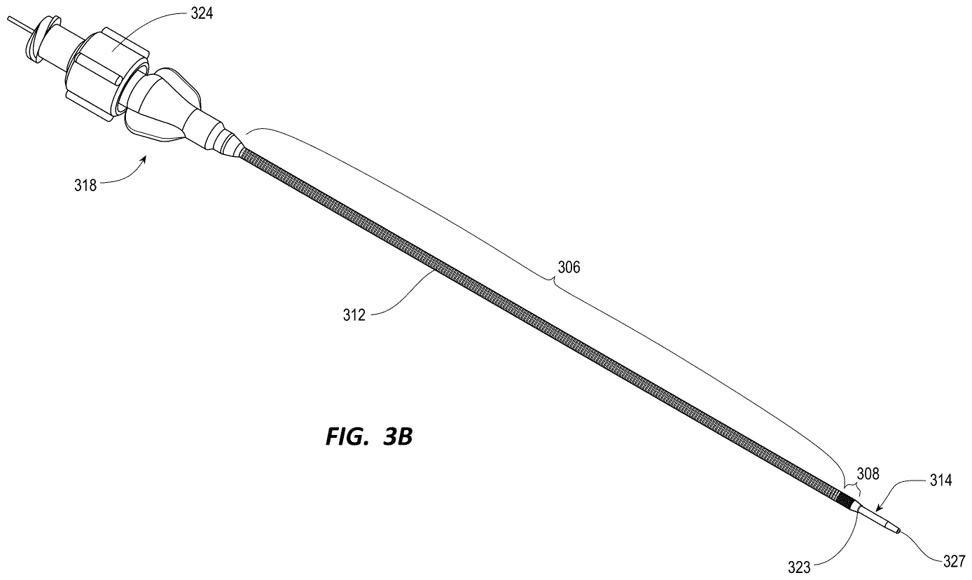

FIG. 3A is a perspective view of an introducer sheath assembly 300 having a catheter sheath 312 (e.g. sheath shaft), a dilator 314, and a guidewire 316. The catheter sheath 312 includes a sheath tube 319 with the coiled wire structure 335 disposed therein. Coiled wire structure 335 can include a first coil and a second coil, as was previously discussed with respect to FIGS. 1A-2.

Coiled wire structure 335 can extend a length of the sheath 312, and include elements as previously described. For example, sheath 312 can include the coiled wire structure 335 having an annealed segment and disposed between a liner and a jacket. Unless otherwise noted, the coiled wire structure 335 may include any aspect (e.g. materials or shape) of the coiled wire structure 104 and/or 234. Furthermore, the liner and the jacket of sheath 312 may include any aspect (e.g., materials or shape) of the liner 108, jacket 105, first coil 102, and/or second coil 106 as seen and described within FIGS. 1A-D.

In some embodiments, the coiled wire structure 335 includes wire disposed more densely in the distal portion 308 than the proximal portion 306 of the coiled wire structure 335. The coiled wire structure 335, including the distal portion 308 and the proximal portion 306, may include any aspect of other coiled wire structures described herein, such as the materials, dimensions, configurations, and positioning of: the coiled wire structure 104, 234 including the proximal portion 138, 238 and the distal portion 136, 236 as described with respect to FIGS. 1A-2.

The introducer sheath assembly 300 can be utilized to facilitate the introduction of a guidewire or catheter into the vasculature or other body cavity of a patient. Micropuncture catheters have been developed to provide a relatively smaller access puncture into the vasculature of the patient. Micropuncture introducer sheath assemblies can include an introducer sheath which allows larger diameter guidewires to be introduced into the vasculature of the patient through the smaller access opening.

In the illustrated embodiment, introducer sheath assembly 300 can include a catheter sheath 312, a dilator 314, and a guidewire 316. During a procedure in which introducer sheath assembly 300 is utilized, catheter sheath 312 and dilator 314 can be coupled together, allowing the introducer sheath assembly 300 to be threaded into the patient. Dilator 314 can be inserted along the length of catheter sheath 312 to provide rigidity and stiffness to facilitate the insertion of catheter sheath 312 into the patient. To insert catheter sheath 312 and dilator 314 into the patient, guidewire 316 can first be inserted into the desired position within the patient through an access needle which has been inserted through the skin of the patient and into a vein, artery, or body cavity. Once guidewire 316 has been threaded through the access needle and into the patient, the needle can be withdrawn leaving guidewire 316 in place within the patient.

The relatively small diameter of the components of a micropuncture introducer sheath assembly 300 can allow for a relatively small access puncture into the patient. For example, typically the access needle utilized with guidewire 316 can be a 21 gauge access needle or smaller. This allows a smaller guidewire 316 to be utilized. For example, guidewire 316 can be a 0.018″ guidewire or smaller. By utilizing such small access needles and guidewires with introducer sheath assembly 300, the original access puncture to the patient's skin, body cavity, or vasculature is quite small. The small access puncture facilitates a shorter recovery time, less trauma to the patient, and while abbreviating the length of bleeding from the access puncture and/or the vasculature of the patient.

Once guidewire 316 is positioned in the desired location within the patient, dilator 314 is threaded over guidewire 316. As previously discussed, dilator 314 is positioned within catheter sheath 312 such that when guidewire 316 is threaded along the length of dilator 314, guidewire 316 is also threaded along the length of catheter sheath 312. Dilator 314 and catheter sheath 312 are then advanced along the length of guidewire 316 through the access puncture in the patient's skin and into the desired position within the patient's body. The configuration of the tip of catheter sheath 312 and dilator 314 result in little tearing or trauma at the access puncture. Instead, catheter sheath 312 and dilator 314 results in stretching of the access puncture in a manner such that the size of the access puncture quickly returns to the original puncture size allowing for quicker healing at the access puncture site. Once dilator 314 and catheter sheath 312 have been inserted into the patient, guidewire 316 is withdrawn from the patient. Subsequently, dilator 314 will be withdrawn from the catheter sheath 312 allowing for the practitioner to access the body cavity or vasculature of the patient through catheter sheath 312.

In one embodiment, a larger diameter guidewire can be threaded through catheter sheath 312 and into the vasculature of the patient without requiring a larger access puncture typically required when a larger guidewire is inserted directly through an access needle. For example, such larger diameter guidewires can be a 0.038″ diameter guidewire which requires an approximately 0.040″ diameter puncture needle to be inserted into the patient. Utilizing a 0.040″ diameter puncture needle instead of the exemplary 21 gauge needle which is utilized in connection with catheter sheath 312 results in an access puncture that can be more than twice the size of the puncture required when utilizing introducer sheath assembly 300.

In the illustrated embodiment, catheter sheath 312 includes sheath hub 318 and sheath tube 319. Sheath tube 319 is coupled to sheath hub 318 in a manner that allows for desired operation of sheath tube 319 relative to sheath hub 318. Additionally, sheath tube 319 can be in fluid communication with sheath hub 318 allowing access to sheath tube 319 along the length of sheath hub 318.

In the illustrated embodiment, sheath hub 318 comprises wings 320a-b and a lower coupler 322. Wings 320a-b facilitate manipulation of catheter sheath 312 and introducer sheath assembly 300 by providing a gripping point for the practitioner during utilization of the introducer sheath assembly 300. Lower coupler 322 is positioned at the proximal end of catheter sheath 312. Lower coupler 322 allows for coupling of the dilator 314 to the catheter sheath 312 during operation of the introducer sheath assembly.

Sheath tube 319 includes a sheath tip 323 positioned at the distal end of the sheath tube 319. Sheath tip 323 is slightly tapered allowing for insertion of the catheter sheath into the access puncture. Sheath tube 319 typically has a resilient and somewhat flexible configuration allowing for introduction of larger diameter guidewires along the length of catheter sheath 312. The resilient nature of sheath tube 319 also permits expansion of the access puncture in the patient's skin while protecting the patient from damage as the guidewire is inserted.

Dilator 314 can include dilator hub 324 and a stiffener tube within dilator sleeve 334 (not labeled in FIG. 3A). A stiffener tube provides additional rigidity and strength to dilator sleeve 334 as introducer sheath assembly 300 is inserted into the patient. Dilator hub 324 allows for manipulation of stiffener tube while also allowing for coupling of the dilator 314 to the catheter sheath 312 during utilization of the introducer sheath assembly 300. In the illustrated embodiment, dilator hub 324 comprises a lower coupler 326 and gripping members 328. Lower coupler 326 is positioned on the distal side of dilator hub 324 allowing for mating engagement of dilator hub 324 and sheath hub 318. Gripping members 328 are positioned on the outside diameter of dilator hub 324 allowing for gripping of the dilator hub 324 by the practitioner. By gripping the dilator hub 324, the practitioner can manipulate the dilator 314 to secure the dilator hub 324 to the sheath hub 318. Additionally, gripping members 328 of dilator hub 324 allow the practitioner to manipulate the introducer sheath assembly 300 during the course of the procedure.

Dilator sleeve 334 includes a dilator tip 327. Dilator tip 327 can include a substantially resilient member which is configured to prevent damage to the patient tissue as introducer sheath assembly 300 is threaded along the length of guidewire 316 and into the patient. Dilator tip 327 is more resilient and deformable than stiffener tube. This is due to the fact that, in embodiments, the stiffener tube can be positioned within dilator sleeve 334 but does not extend into dilator tip 327.

Transition 329 represents the point at which the stiffener tube terminates and dilator tip 327 begins. In the illustrated embodiment, transition 329 is positioned at a point that is more proximal than traditional transition points on stiffened micropuncture catheters. This allows for transition 329 to be positioned proximally to the sheath tip 323 of catheter sheath 312 when dilator 314 is threaded along the length of catheter sheath 312 and dilator hub 324 is coupled to sheath hub 318. Additionally, dilator tip 327 has a greater length from transition 329 to the distal tip of dilator tip 327. The greater length of dilator tip 327 allows a portion of dilator tip 327 to be positioned proximally to sheath tip 323 and within the catheter sheath 312. Additionally, a portion of dilator tip 327 can be positioned outside of catheter sheath 312 and distally to sheath tip 323.

The positioning of transition 329 within catheter sheath 312 allows catheter sheath 312 to provide strain relief to dilator tip 327 subsequent to lateral movement of the dilator tip 327. Positioning transition 329 within catheter sheath 312 relieves the strain that would normally be carried primarily at transition 329. Such strain is caused due to the stiffer configuration of dilator sleeve 334, which is co-extensive with the stiffener tube, and the more flexible nature of dilator tip 327. By providing strain relief subsequent to lateral movement of dilator tip 327, potentially damaging forces at transition 329 are dissipated. By dissipating such forces, kinking, buckling, or bending of catheter tip 327 at transition 329 is minimized in a manner that could result in the failure of dilator tip 27 during the procedure. In other words, minimizing the potential for damage at transition 329 provides for continued integrity of dilator 314 during the course of an insertion procedure.

As will be appreciated by those skilled in the art, a variety of types and configurations of introducer sheath assemblies can be utilized without departing from the scope or spirit of the present invention. For example, in one embodiment, the transition point is positioned at a traditional location along the length of the dilator. An elongate catheter sheath is provided such that the sheath tip is positioned distally to the transition. In another embodiment, a standard sized catheter sheath is utilized with a dilator having a shorter stiffener. An elongated dilator tip is provided such that the transition is moved proximally behind the tip of the catheter sheath. In yet another embodiment, a combination of an elongated catheter sheath, an elongated dilator tip, and a proximally positioned transition is utilized to provide a strengthened dilator tip. In yet another embodiment, the dilator hub is secured to the sheath hub utilizing other than a lower coupling. In yet another embodiment, a single hub is provided instead of two hubs.

FIG. 3B is a perspective view of the introducer sheath assembly of FIG. 3A, assembled for use. In the illustrated embodiment, the introducer sheath assembly can include a catheter sheath 312 (e.g. sheath shaft), a dilator 314, and a guidewire (not labeled in FIG. 3B), as was described with respect to FIG. 3A.

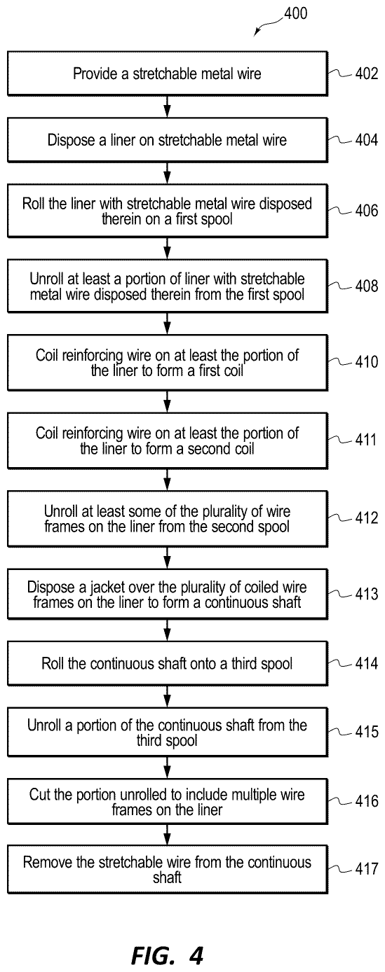

FIG. 4 is a flow diagram of an example of a method 400 of manufacturing a sheath, such as an introducer sheath, according to one embodiment of the present disclosure. The steps shown in FIG. 4 may be optional and not necessarily including in each process. Further, various steps may be completed in different sequences from those shown in FIG. 4.

At block 402, method 400 can include providing a stretchable wire (e.g., a mandrel). The method 400 also may include providing a liner having the stretchable wire disposed therein or disposing a liner on the stretchable metal wire (at block 404). For example, method 400 may include disposing the liner on the metal wire. In some embodiments, disposing the liner on the stretchable metal wire can include sliding the liner onto the stretchable metal wire.

At block 406, method 400 can include rolling the liner with the stretchable metal wire disposed therein (e.g., the elongate member 122 of FIGS. 1C-D) on a first spool. For example, at least 1000 feet of the liner with the stretchable wire disposed therein may be rolled onto the first spool.

At block 408, method 400 can include unrolling at least a portion of the liner with stretchable metal wire disposed therein from the first spool.

At block 410, method 400 includes coiling the reinforcing wire over the liner and/or stretchable metal wire to form a first coil of the coiled wire structure or coiled wire structure. In embodiments, the first coil can include a distal portion and a proximal portion. The wire may be coiled or disposed more densely in the distal portion than in the proximal portion, and the segment of the reinforcing wire that is annealed may be at least proximate to the distal portion of the coiled wire structure. In some embodiments, the annealed segment of the reinforcing wire is coiled over the liner within about 60 seconds of annealing the segment of the reinforcing wire.

At block 411, method 400 can include coiling the reinforcing wire over the liner and first coil to form a second coil of the coiled wire structure or coiled wire structure. The second coil can be formed over the first coil, and can be coiled in an opposite direction as the first coil. Otherwise state, the first coil can be coiled around the elongate member as the elongate member is translating in a first direction, and the second coil can be coiled around the elongate member as the elongate member is translating in a second direction, or opposite the first direction. Similar to the first coil, the second coil can include a distal portion and a proximal portion. The wire of the second coil may be coiled or disposed more densely in the distal portion than in the proximal portion, and the segment of the reinforcing wire that is annealed may be at least proximate to the distal portion of the coiled wire structure. In some embodiments, the annealed segment of the reinforcing wire is coiled over the liner and first coil within about 60 seconds of annealing the segment of the reinforcing wire.

In embodiments, block 411 can optionally include unrolling at least a portion of the liner with stretchable metal wire disposed therein from the second spool to the first spool. In embodiments, block 411 can optionally include unrolling at least a portion of the liner with stretchable metal wire disposed therein opposite as was performed at block 408.

In some embodiments, block 410 and/or block 411 can optionally include, annealing a segment of the reinforcing wire such that the annealed segment of the reinforcing wire is between two unannealed regions of the reinforcing wire. In some embodiments, annealing the segment of the reinforcing wire can include activating one or more electrodes positioned along the reinforcing wire between the spool and the liner (e.g., the elongate member) to heat the segment of the reinforcing wire, and then deactivating the one or more electrodes such that the two unannealed regions of the reinforcing wire bordering the segment of reinforcing wire are not heated by the one or more electrodes. As described above, the one or more electrodes can be secured to one or more pulleys positioned along the reinforcing wire between the spool and the liner. Annealing the segment of the reinforcing wire may include annealing the segment of the reinforcing wire between two pulleys (e.g., pulleys 116A-B of FIGS. 1C-D) positioned along the reinforcing wire between the spool and the liner, with the one or more electrodes being secured to at least one pulley (e.g., both) of the two pulleys. In some embodiments, annealing the segment of the reinforcing wire includes bringing the segment of the reinforcing wire to at least about 75% of the melting temperature of the reinforcing wire, such as about 75% to about 85%, about 75% to about 80%, about 77.5% to about 82.5%, about 80% to about 85%, about 75%, about 77.5%, about 80%, about 82.5%, or about 85% of the melting temperature of the reinforcing wire.

In many embodiments, annealing the segment of the reinforcing wire reduces the at least one (e.g., both) of the hardness and/or spring constant in the coils of the coiled wire structure (or coiled wire structure) that includes the segment of the reinforcing wire that is annealed. For example, the hardness and/or spring constant in the coils of the coiled wire structure that include the segment of the reinforcing wire that is annealed may be reduced about 10% to about 20%, about 10% to about 15%, about 12.5% to about 17.5%, about 15% to about 20%, at least about 10%, at least about 12.5%, at least about 15%, at least about 17.5%, about 10%, about 12.5%, about 15%, about 17.5%, or about 20% relative to portions of the coiled wire structure or structure that are not annealed.

In some embodiments, block 410 and/or block 411 can include pulling the elongate member from the first spool and/or second spool as the reinforcing wire is being coiled around a portion of the elongate member (e.g., as at least one of the first spool 112 and/or the one or more pulleys 116A-D are rotated around the elongate member 122, as seen in FIGS. 1A-D). For example, method 400 may include pulling the elongate member from the first spool and/or second spool effective to form the proximal portion and the distal portion of the coiled wire structure on the elongate member. More specifically, the elongate member may be pulled from the first spool and/or second spool at a first rate when the proximal portion is being formed and a second rate slower than the first rate when the distal portion is being formed. Pulling the elongate member from the first spool and/or second spool at the first rate and the second rate is effective for form the distal portion having the reinforcing wire coiled more densely thereon than the proximal portion of the coiled wire structure.

In embodiments, method 400 may also include heating the liner having the coiled wire structure 104 disposed thereon prior to or as the liner and the coiled wire structure, and elongate member are rolled onto the second spool. This heating may help tack the coiled wire structure 104 to the surface of the liner 108.

At block 412, the method 400 can include unrolling a length of the coiled wire structure and liner on the elongate member from the first and/or second spool.

At block 413, the method 400 can include and securing or disposing a jacket over one or more of the unrolled coiled wire structure and the liner. Securing or disposing the jacket over the unrolled coiled wire structure and liner may include sliding the jacket over the unrolled coiled wire structure and the liner, and then heat shrinking the jacket over the unrolled coiled wire structures and the liner. In some embodiments, the liner 108 may be heated before the jacket 105 is disposed over the coiled wire structure and the liner.

At block 413, method 400 may optionally include, before disposing the jacket over the coiled wire structure and the liner, sliding a heat shrink material over at least some of the reinforcing wire coiled at a proximal region of the liner, and also heat shrinking the heat shrink material over the at least some of the reinforcing wire coiled at the proximal region of the liner. In some embodiments, the method 400 includes reflowing the liner and the jacket after securing the jacket over the coiled wire structures and the liner.

At block 414, the method 400 may include rolling the coiled wire structure onto a third spool.

At block 415, the method 400 may include unrolling a portion of the elongate member including coiled wire structure(s) from the third spool.

At block 416, the method 400 may include cutting the unrolled portion of the elongate member including the coiled wire structure(s) from the third spool. For example, the portion cut from the third spool may be approximately three feet of continuous sheath 140 including a coiled wire structure between the liner and the jacket.

At block 417, the method 400 also may include removing the metal wire or mandrel from the liner 108 after cutting the portion of the continuous shaft from the third spool. For example, method 400 may include stretching the metal wire while the metal wire is within the liner to narrow the metal wire, and then pulling the metal wire from the liner.

Optionally, at the outset of method 400, method 400 can further include cutting or isolating a portion of a continuous shaft removed from the third spool to form a first sheath. For example in some embodiments, discrete sheaths of a pre-determined length can be formed via the above method. In alternate embodiments, multiple sheaths, including multiple braided structures can be disposed continuously. In such cases, discrete sheaths may need to be cut and/or isolated. Method 400 can further include removing at least a portion of the reinforcing wire from the first sheath, and may include laser cutting or laser ablating the coiled wire structure about 0.5 mm to about 2 mm from a terminating end of the sheath.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “near.” For each such reference, it is to be understood that, in some embodiments, the value, feature, or characteristic may be specified without approximation. For example, where qualifiers such as “near” and “approximately” are used, these terms include within their scope the qualified words in the absence of their qualifiers. For example, where the term “approximately aligned” is recited with respect to a feature, it is understood that in further embodiments, the feature can have a precisely aligned configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A method of manufacturing a sheath, the method comprising:
providing an elongate member comprising a liner disposed on an outer surface of a wire;
disposing the elongate member such that a portion of the elongate member is suspended between a first support point and a second support point;
translating the suspended portion of the elongate member in a first direction;
disposing a first coil of a reinforcing wire onto the suspended portion of the elongate member as the suspend portion is translated in the first direction;
translating the suspend portion in a second direction; and
disposing a second coil of the reinforcing wire onto the suspended portion as the suspend portion is translated in the second direction.

2. The method of claim 1, wherein disposing a first coil of a reinforcing wire onto the suspended portion of the elongate member and disposing a second coil of a reinforcing wire onto the suspended portion of the elongate member comprises coiling the reinforcing wire around an outer circumferential surface of the elongate member.

3. The method of claim 1, wherein the second coil is disposed over the first coil.

4. The method of claim 1, wherein the second coil coils around the elongate member in a direction opposite the first coil.

5. The method of claim 1, wherein the wire of the elongate member comprises a silver plated copper wire.

6. The method of claim 1, wherein translating the suspended portion in the first direction comprises rotating a first spool and a second spool in a first rotational direction; wherein translating the suspended portion in the second direction comprises rotating the first spool and the second spool in a second rotational direction.

7. The method of claim 1, wherein translating the suspended portion in the first direction comprises translating a first chuck and a second chuck in a first rotational direction; wherein translating the suspended portion in the second direction comprises translating the first chuck and the second chuck in a second rotational direction.

8. The method of claim 1, further comprising:
removing the wire of the elongate member from the liner; and
securing a jacket over remaining liner, the first coil, and the second coil.

9. The method of claim 1, further comprising annealing a segment of the reinforcing wire such that the segment is between two unannealed regions of the reinforcing wire.

10. The method of claim 9, wherein annealing the segment of the reinforcing wire includes:
activating one or more electrodes positioned between a third spool comprising the reinforcing wire and the liner to heat the segment of the reinforcing wire; and
deactivating the one or more electrodes such that the two unannealed regions of the reinforcing wire border the segment are not heated by the one or more electrodes.

11. The method of claim 10, wherein the one or more electrodes are secured to one or more pulleys positioned between the third spool and the liner.

12. The method of claim 11, wherein annealing the segment of the reinforcing wire includes annealing the segment of the reinforcing wire between two pulleys positioned between the third spool and the liner, the one or more electrodes being secured to at least pulley of the two pulleys.

13. The method of claim 9, wherein annealing the segment of the reinforcing wire reduces a hardness and/or spring constant by at least about 10% in coils of the first coil or the second coil that include the segment of reinforcing wire that is annealed relative to the hardness and/or spring constant of coils of first coil or second coil that include the two unannealed regions.

14. A sheath comprising:
an inner layer defining a lumen of the sheath;
a coiled wire structure disposed on an exterior circumferential surface of the inner layer, the coiled wire structure comprising:
a first coil comprising a reinforcing wire coiled in a first direction; and
a second coil comprising a reinforcing wire coiled in a second direction;
an outer layer disposed on the exterior circumferential surface of the coiled wire structure.

15. The sheath of claim 14, further comprising a proximal portion and a distal portion, wherein the distal portion of the sheath comprises a portion of the coiled wire structure that is more densely coiled than a portion of the coiled wire structure corresponding to the proximal portion.

16. The sheath of claim 14, wherein the first coil is disposed beneath the second coil.

17. The sheath of claim 14, wherein a portion of the first coil is coiled more densely than a portion of the second coil.

18. The sheath of claim 14, wherein a portion of the coiled wire structure comprises a segment of annealed reinforcing wire.

19. A system for coiling reinforcing wire around a liner, the system comprising:
a first spool having liner rolled thereon, the liner having a metal wire disposed therein;
a spool of reinforcing wire;
a coiling assembly configured to:
coil the reinforcing wire on a portion of the liner as the liner is unrolled from the first spool to form a first coil of a coiled wire structure; and
coil the reinforcing wire on the portion of the liner unrolled from the first spool as it is rolled onto the first spool to form a second coil of a coiled wire structure;
a jacket assembly configured to secure a jacket over the coiled wire structure coiled on the portion of the liner; and
an additional spool positioned to roll the coiled wire structure coiled on the portion of the liner thereon.

20. The system of claim 19, further comprising one or more pulleys positioned between the spool of reinforcing wire and the liner and configured to receive an electrical charge effective to anneal a segment of the reinforcing wire before the reinforcing wire is coiled around the liner.
